(19) United States
(12) Patent Application Publication
(10) Pub. No.: US 2005/0164584 A1
(43) Pub. Date: Jul. 28, 2005

(54) Retractable nonwoven layers having minimal application of coalesced elastomers

(76) Inventors: Stephen Avedis Baratian, Roswell, GA (US); Phillip A. Schorr, Atlanta, GA (US); Arthur Edward Garavaglia, Alpharetta, GA (US); Susan Carol Sudbeck, Alpharetta, GA (US); Dan K. Schiffer, Mt. Pleasant, SC (US); Mark Bruce Majors, Cumming, GA (US); Kimberly Nicole Sanders, Woodstock, GA (US); David Lewis Myers, Cumming, GA (US); Glen Thomas Mildenhall, Marietta, GA (US); Oomman Painummoottil Thomas, Alpharetta, GA (US)

Correspondence Address: Roland W. Norris;Pauley Petersen & Erickson, Suite 365, 2800 West Higgins Road, Hoffman Estates, IL, 60195, US

(21) Appl. No.: 10/750,535

(22) Filed: Dec. 31, 2003

(51) Int. Cl. B32B 5/12; B32B 27/14; D04H 3/00; D04H 1/00; D04H 3/16; A61F 13/20; A61F 13/15; D04H 13/00; D04H 5/00; B05D 5/00; B32B 3/00

(52) U.S. Cl. 442/329; 428/114; 428/196; 442/401; 604/385.01; 604/385.24; 427/286

(57) ABSTRACT

An extensible fluid permeable substrate having at least one direction of extensibility in an X-Y plane is provided with improved retraction to make the composite material suitable for disposable garment applications with minimal application of coalesced elastomeric materials. A pattern of untensioned coalesced elastomeric stripes is applied on an X-Y plane surface of the substrate in low add-on amounts of between about 20% to about 100% of the substrate basis weight to make the composite material. The longitudinal axes of the coalesced elastomeric stripes are oriented substantially along the direction of substrate extensibility and desired retraction of the composite material. By applying the minimal amount of elastomer necessary in an open pattern, the economical composite material also avoids negative by-products of elastic coatings or films such as bad hand, bad drape, loss of fluid transfer or intake ability and lack of breathability. In a particular embodiment the coalesced elastomers are applied as electrospun microfibers yielding a very low add-on rate with fluid permeable coalesced elastomer areas while providing improved retractive properties to the substrate.

### BACKGROUND OF THE INVENTION

It is desirable that personal care absorbent articles, and especially garments such as diapers, training pants, or incontinence garments, without limitation referred to generically now for ease of explanation as “diapers,” provide a close, comfortable fit about body of the wearer and contain body exudates while maintaining skin health such as through breathability of the garment. In certain circumstances, it is also desirable that such garments are capable of being pulled up or down over the hips of the wearer to allow the wearer or care giver to easily pull the article on and easily remove the article.

The person having ordinary skill in the art of disposable diaper manufacture will appreciate that the disposable diaper is generally made up of the layers of a substantially liquid-impermeable backsheet or outer cover, a liquid-permeable topsheet or liner, and a liquid retention structure or absorbent core located between the backsheet and the liner. Often, these layers, especially with regard to the liners and some components of outer covers, comprise a nonwoven which can economically be made extensible but which lacks sufficient retraction.

Great attention has particularly been applied to the so called “cuff areas” of the waist band and leg holes. However it is now considered optimal in some garment applications to have entire substrates, e.g. liners and outer covers, which have extensible and retractive abilities. Various schemes for producing elastic or retractive materials for disposable diapers have been proposed. Unfortunately, application of elastic or elastomeric materials to the nonwoven webs to gain elasticity is expensive and may have various shortcomings including fluid barrier problems such as lack of liquid transmission or lack of vapor breathability, loss of good hand, drape, and appearance, difficulty in handling monolithic elastic elements, etc., when considered in light of certain garment layer applications, particularly liners and, in some instances, layers within an outer cover assembly.

Thus, there further remains a need in the art to provide ease and economy of manufacture of retractive garment layers, especially where such garments are intended to be disposable.

### SUMMARY OF THE INVENTION

The present invention provides for precise and limited amounts of a coalesced elastomer deposition on an extendible substrate web thereby creating an economical composite material, sometimes simply referred to as a “composite,” providing an improved retractive force for the web. The composite will, when used in a garment, help create a conformable fit and improved functionality of the article to the wearer, while maintaining economy of manufacture, and improved retractive performance for the substrate layer. For example, a liner of the composite material will minimize its wrinkling if the garment or article undergoes stretch and relaxation due to movement of the wearer. The avoidance of wrinkling will better enable the liner material to achieve its functionality of handling exudate insults from the wearer. The composite will retain the essential fluid handling characteristics desired for certain layers within the garment such as fluid transmission, of liquid or vapor, or both, which would ordinarily be restricted by an elastic layer. In some aspects of the invention a pattern of untensioned coalesced elastomeric stripes is applied on an X-Y plane surface of the substrate in an add-on amount of between about 20% to about 100% by weight, to make the retractive composite material. Aspects of the present invention may provide for the placement of coalesced elastomers areas in open patterns having stripes with longitudinal axes oriented to one or more directions of web extensibility.

One aspect of the present invention provides for the printing or spraying application (hereinafter referred to collectively as “printing”) of emulsions or solvents of elastomer onto an extensible substrate of nonwoven web material useful for a back sheet or liner in a disposable absorbent article. The elastomer material coalesces to form an area of improved retraction as the carrier fluid of the elastomer is dispersed, e.g., dries or evaporates. In another aspect of the invention the coalesced elastomers are applied to the substrate via electrospinning as charged microfibers onto a grounded or oppositely charged substrate. A “microfiber” as referred to herein will typically be a fiber of between 50 nanometers to 5 microns, and desirably about 500 nanometers to 3 microns, in size. The microfiber or microfibers coalesce into areas of improved retraction as the coalesced elastomeric fiber or fibers accumulate on the substrate surface. In another aspect, the coalesced elastomers may serve as a component of a biaxially extendible backsheet, i.e., a backsheet extendible in both longitudinal and lateral axes of the web. In another aspect, the coalesced elastomer application does not interfere with liquid uptake or vapor transmission of the composite web due to its open, and in some instances fibrous, nature.

Generally, an exemplary embodiment of the present invention may suitably provide integral members of a disposable absorbent article in the form of a garment comprising a front waist section, a rear waist section, an intermediate section which extends between and connects the waist sections, a pair of laterally opposed side edges, a pair of longitudinally opposed waist edges, a longitudinal direction and a lateral direction. Extendible backsheet and liner materials according to the present invention can be provided as economical retractive materials with fluid transmission ability that retain the ability to conform to the body of the wearer. Pant-like disposable absorbent garments with waist and leg cuff areas may be provided having at least one of the waist and leg cuff areas comprising a retractive composite according to the present invention.

### BRIEF DESCRIPTION OF THE DRAWINGS

The present invention will be more fully understood and further advantages will become apparent when reference is made to the following detailed description of the invention and the drawings, in which:

Table 1 details the data from testing of control samples and four embodiments of screen printed samples of the present invention.

Table 2 details the data from testing of six embodiments of electrospinning samples of the present invention.

Table 3 is a synopsis of mechanical property improvements shown by examples of screen printing aspects of the present invention.

Table 4 is a synopsis of mechanical property improvements shown by examples of electrospinning aspects of the present invention.

FIG. 1 shows a partially cut-away, top plan view of the inward surface of an example of a garment according to one aspect of the invention.

FIG. 2 illustrates an electrospinning manufacturing technique for the low weight application of coalesced elastomer fibers to an extensible web according to the present invention

FIGS. 3-5 illustrate patterns of coalesced elastomer suitably applied to the extensible web substrate.

FIGS. 6-12 are graphs showing certain performance characteristics of the present invention.

FIGS. 13-18 illustrate test apparatus for measuring fluid uptake of a material.

### DEFINITIONS

As used herein, the terms “elastic”, “elastomeric”, and forms thereof, mean any material which, in its final form in the completed diaper, upon application of a biasing force, is stretchable, that is, elongatable, and which will return towards substantially its original shape with force upon release of the stretching, elongating force. The terms “extendible” and “extensible” refer to a material which is stretchable in at least one direction but which may or may not have sufficient recovery to be considered elastic.

“Hysteresis” as used herein refers to material recovery after stretch with zero percent being a perfect return or complete recovery of the retractive material while 100% loss would indicate that no recovery was made and hence the material tested is not retractive.

“Immediate set” as used herein refers to permanent plastic deformation of the material. For example a 10 cm piece of material when stretched to 15 cm and allowed to relax may return to only 12 cm, for a gain in length of 2 cm or a 20% immediate set.

As used herein, the term “machine direction”, or MD, means the length of a fabric in the direction in which it is produced. The term “cross machine direction” or CD means the width of fabric, i.e. a direction generally perpendicular to the MD. As described in the X, Y and Z axes, X will be MD, Y will be CD and Z will be depth or thickness of the material.

As used herein, the term “nonwoven web” or “nonwoven material” means a web having a structure of individual fibers, filaments or threads which are interlaid, but not in a regular or identifiable manner such as those in a knitted fabric or films that have been fibrillated. Nonwoven webs or materials have been formed from many processes such as, for example, meltblowing processes, spunbonding processes, and bonded carded web processes. The basis weight of nonwoven webs or materials is usually expressed in ounces of material per square yard (osy) or grams per square meter (gsm), and the fiber diameters usable are usually expressed in microns. (Note that to convert from osy to gsm, multiply osy by 33.91.) The term “fabrics” is used to refer to all of the woven, knitted and nonwoven fibrous webs.

As used herein, the term “neck” or “neck stretch” interchangeably means that the fabric is extended under conditions reducing its width or its transverse dimension. The controlled extension may take place under cool temperatures, room temperature or greater temperatures and is limited to an increase in overall dimension in the direction being extended up to the elongation required to break the fabric. The necking process typically involves unwinding a sheet from a supply roll and passing it through a brake nip roll assembly driven at a given linear speed. A take-up roll or nip, operating at a linear speed higher than the brake nip roll, extends the fabric and generates the tension needed to elongate and neck the fabric. U.S. Pat. No. 4,965,122, to Morman, incorporated by reference in its entirety, discloses a process for providing a reversibly necked nonwoven material which may include necking the material, then heating the necked material, followed by cooling.

As used herein, the term “neckable material or layer” means any material which can be necked such as a nonwoven, woven, or knitted material. As used herein, the term “necked material” refers to any material which has been extended in at least one dimension, (e.g. lengthwise), reducing the transverse dimension, (e.g. width), such that when the extending force is removed, the material can be pulled back, or relax, to its original width. The necked material typically has a higher basis weight per unit area than the un-necked material. When the necked material returns to its original un-necked width, it should have about the same basis weight as the un-necked material. This differs from stretching/orienting a material layer, during which the layer is thinned and the basis weight is permanently reduced.

Typically, such necked nonwoven fabric materials are capable of being necked up to about 80 percent. For example, the neckable backsheet 30 of the various aspects of the present invention may be provided by a material that has been necked from about 10 to about 80 percent, desirably from about 20 to about 60 percent, and more desirably from about 30 to about 50 percent for improved performance. For the purposes of the present disclosure, the term “percent necked” or “percent neckdown” refers to a ratio or percentage determined by measuring the difference between the pre-necked dimension and the necked dimension of a neckable material, and then dividing that difference by the pre-necked dimension of the neckable material and multiplying by 100 for percentage. The percentage of necking (percent neck) can be determined in accordance with the description in the above-mentioned U.S. Pat. No. 4,965,122.

Words of degree, such as “about”, “substantially”, and the like used herein in the sense of “at, or nearly at when given the manufacturing, testing, and material tolerances inherent in the stated circumstances” and are used to prevent the unscrupulous infringer from unfairly taking advantage of the invention disclosure where exact or figures or absolutes are stated as an aid to understanding the invention.

### DETAILED DESCRIPTION OF THE PRESENTLY PREFERRED EMBODIMENTS

The various aspects and embodiments of the invention will be described in the context of disposable absorbent articles, and more particularly referred to, without limitation and by way of illustration only, as a disposable diaper. It is, however, readily apparent that the present invention could also be employed with other absorbent articles, such as feminine care articles, various incontinence garments, medical garments, and any other disposable garments, whether absorbent or not, needing an easily conformable structure. Typically, the disposable articles or garments are intended for limited use and are not intended to be laundered or otherwise cleaned for reuse. A disposable diaper, for example, is discarded after it has become soiled by the wearer.

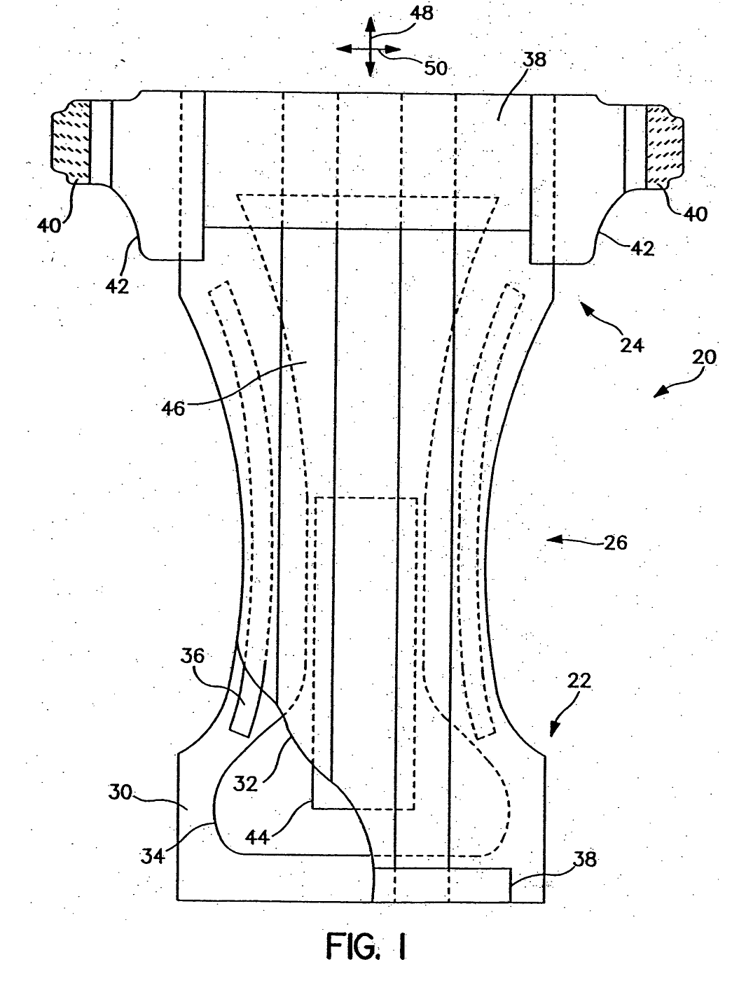

FIG. 1 is a representative plan view of an absorbent article, such as disposable diaper 20, of the present invention in its flat-out, or unfolded state. Portions of the structure are partially cut away to more clearly show the interior construction of the diaper 20. The surface of the diaper 20 which contacts the wearer is facing the viewer.

With reference to FIG. 1, the disposable diaper 20 generally defines a front waist section 22, a rear waist section 24, and an intermediate section 26 which interconnects the front and rear waist sections. The front and rear waist sections 22 and 24 include the general portions of the article which are constructed to extend substantially over the wearer's front and rear abdominal regions, respectively, during use. The intermediate section 26 of the article includes the general portion of the article that is constructed to cover the wearer's crotch region and extend between the legs. Thus, the intermediate section 26 is an area where repeated liquid surges typically occur in the diaper or other disposable absorbent article.

The diaper 20 includes, without limitation, an outer cover, or backsheet 30, a liquid permeable bodyside liner, or topsheet, 32 positioned in facing relation with the backsheet 30, and an absorbent body, or liquid retention structure 34, such as an absorbent pad, which is located between the backsheet 30 and the liner 32. The backsheet 30 defines a length, or longitudinal direction 48, and a width, or lateral or transverse, direction 50 which, in the illustrated embodiment, coincide with the length and width of the diaper 20. The liquid retention structure 34 generally has a length and width that are less than the length and width of the backsheet 30, respectively. Thus, marginal portions of the diaper 20, such as marginal sections of the backsheet 30, may extend past the terminal edges of the liquid retention structure 34. In the illustrated embodiments, for example, the backsheet 30 extends outwardly beyond the terminal marginal edges of the liquid retention structure 34 to form side margins and end margins of the diaper 20. The topsheet 32 is generally coextensive with the backsheet 30 but may optionally cover an area which is larger or smaller than the area of the backsheet 30, as desired.

To provide improved fit and to help reduce leakage of body exudates from the diaper 20, the diaper side margins and end margins may be elasticized with added monolithic elastic members, as known in the art. For example, as representatively illustrated in FIG. 1, the diaper 20 may include leg elastics 36 which are constructed to operably tension the side margins of the diaper 20 to provide elasticized leg bands which can closely fit around the legs of the wearer to reduce leakage and provide improved comfort and appearance. Waist elastics 38 can be employed to elasticize the end margins of the diaper 20 to provide elasticized waistbands. The waist elastics can be configured to provide a resilient, comfortably close fit around the waist of the wearer.

As is known, fastening means, such as hook and loop fasteners, with a hook portion shown at ref. no. 40, may be employed to secure the diaper 20 on a wearer. Alternatively, other fastening means, such as buttons, pins, snaps, adhesive tape fasteners, cohesives, fabric-and-loop fasteners, or the like, may be employed. In the illustrated embodiment, the diaper 20 includes a pair of side panels 42 to which the fasteners 40 are attached. Generally, the side panels 42 are attached to the side edges of the diaper 20 in one of the waist sections and extend laterally outward therefrom. The side panels 42 may be elasticized.

The diaper 20 may also include a surge management layer 44, located between the topsheet 32 and the liquid retention structure 34, to rapidly except fluid exudates and distribute the fluid exudates to the liquid retention structure 34 within the diaper 20. Examples of surge management layers 44 are described in U.S. Pat. No. 5,486,166 to Bishop and U.S. Pat. No. 5,490,846 to Ellis, both of which are incorporated herein by reference in their entirety.

As representatively illustrated in FIG. 1, the disposable diaper 20 may also include a pair of containment flaps 46 which are configured to provide a barrier to the lateral flow of body exudates. The containment flaps 46 may be located along the laterally opposed side edges of the diaper 20 adjacent the side edges of the liquid retention structure 34. Each containment flap 46 typically defines an unattached edge which is configured to maintain an upright, perpendicular configuration in at least the intermediate section 26 of the diaper 20 to form a seal against the wearer's body. The containment flaps 46 may extend longitudinally along the entire length of the liquid retention structure 34 or may only extend partially along the length of the liquid retention structure 34. When the containment flaps 46 are shorter in length than the liquid retention structure 34 the containment flaps 46 can be selectively positioned anywhere along the side edges of the diaper 20 in the intermediate section 26. Such containment flaps 46 are generally well known to those skilled in the art. For example, suitable constructions and arrangements for containment flaps 46 are described in U.S. Pat. No. 4,704,96 to K. Enloe, which is incorporated herein by reference in its entirety.

The diaper 20 may be of various suitable shapes. For example, the diaper may have an overall rectangular shape, T-shape or an approximately hour-glass shape. Other suitable components which may be incorporated on absorbent articles of the present invention may include waist flaps and the like which are generally known to those skilled in the art. Examples of diaper configurations suitable for use in connection with the instant invention which may include other components suitable for use on diapers are described in U.S. Pat. No. 4,798,603 to Meyer et al.; U.S. Pat. No. 5,176,668 to Bernardin; U.S. Pat. No. 5,176,672 to Bruemmer et al.; U.S. Pat. No. 5,192,606 to Proxmire et al., and U.S. Pat. No. 5,509,915 to Hanson et al., which are incorporated herein by reference in their entirety.

The various components of the diaper 20 can be integrally assembled together employing various types of suitable attachment means, such as adhesive, ultrasonic bonds, thermal bonds, or combinations thereof. In one embodiment, for example, the liner 32 and backsheet 30 may be assembled to each other with the waist and leg area elastomeric adhesives, such as the hot melt, pressure-sensitive elastomeric adhesive, which thereby serves as both the elastic members 36 and 38, and at least a part of the attachment mechanism.

As known in the art, the backsheet 30 generally includes a fabric or material layer which may be operatively attached-or otherwise joined to the other diaper layers to extend over a major portion of the outward surface of the article. The material for a backsheet 30 is generally selected for superior feel, light weight, liquid impermeability, vapor permeability, and inherent hook attachment acceptance in a desirable embodiment of the present invention. An outer surface component of an extendable backsheet assembly for the garment may be produced according to the present invention with a retractive spunbond facing serving as a fastening material for fabric loop type fasteners and/or providing a more cloth-like feel made more retractive through the techniques of the present invention. One such material may be a 0.5 or 0.6 osy spunbond nonwoven comprising polypropylene fibers.

Desirably, the backsheet 30 is constructed to be permeable to at least water vapor.; For example, in particular embodiments, the backsheet 30 may define a water vapor transmission rate (WVTR) according to the Mocon Water Vapor Transmission Rate Test of about 400 g/sq.m/24 hr. to about 3000 g/sq.m/24 hr. Materials which have a WVTR less than those above may not allow a sufficient amount of water vapor diffusion out of the diaper and undesirably result in increased levels of skin hydration. A Mocon WVTR test is described in U.S. Pat. No. 6,156,421 to Stopper et al. which is incorporated by reference in its entirety. Use of a composite according to the present invention is not believed to significantly impede water vapor transmission when used as a component of a backsheet or outer cover assembly.

The liner 32, as representatively illustrated in FIG. 1, desirably presents a body-facing surface that is compliant, soft-feeling, and non-irritating to the wearer's skin. Further, the liner 32 can be less hydrophilic than the liquid retention structure 34, and is sufficiently porous to be liquid permeable, permitting liquid to readily penetrate through its thickness to reach the absorbent composite. The liner layer 32 is typically employed to help isolate the wearer's skin from liquids held in the liquid retention structure 34.

Various woven and nonwoven fabrics or extruded or blown films might be used for the liner 32. For example, the liner 32 may be composed of a meltblown or spunbond web of substantially continuous polymer fibers, and may also be a bonded-carded-web. Layers of different materials that may have different fiber deniers can also be used. The various fabrics can be composed of natural fibers, synthetic fibers or combinations thereof. The liner 32 may be composed of a substantially hydrophobic material, and the hydrophobic material may optionally be treated with a surfactant or otherwise processed to impart a desired level of wettability and hydrophilicity. In a particular embodiment of the invention, liner 32 can be a nonwoven, spunbond polypropylene fabric composed of about 1.0-5.0 denier fibers formed into a web having a basis weight desirably of about 0.6 osy and density of about 0.065 g/cc.

In some applications of disposable absorbent article construction it is desirable that both the backsheet 30 and the liner 32 are extendible in at least the lateral direction for improved fit and performance of the garment. Desirably, the liner 32 generally comprises extendible materials for compatibility with the backsheet 30 as well as for reduced cost and improved manufacturing efficiency. Extendible materials by themselves may lack the desired or necessary retractability and thus will benefit from the present invention. Suitable extendible materials for use with the present invention may include nonwoven webs, woven materials and knitted materials, extruded films, blown foams, or the like. Such webs should be compatible with the applied coalesced elastomers according to the dictates of the present invention.

Nonwoven fabrics or webs have been formed from many processes, for example, bonded carded web processes, meltblown processes and spunbond processes. The extendible material may be formed from at least one member selected from fibers and filaments of inelastic polymers. Such polymers include polyesters, for example, polyethylene terephthalate, polyolefins, for example, polyethylene and polypropylene, polyamides, for example, nylon 6 and nylon 66. These fibers or filaments may be used alone or in mixtures of two or more of the polymers. Suitable fibers may also include natural and synthetic fibers as well as bicomponent, multi-component, and shaped polymer fibers. Many polyolefins are available for fiber production according to the present invention, for example, fiber forming polypropylenes include Exxon Chemical Company's Escorene7 PD 3445 polypropylene and Himont Chemical Company's PF-304. Polyethylenes such as Dow Chemical's ASPUN7 6811A linear low density polyethylene, 2553 LLDPE and 25355 and 12350 high density polyethylene are also suitable polymers. The nonwoven web layer may be bonded to impart a discrete bond pattern with a prescribed bond surface area.

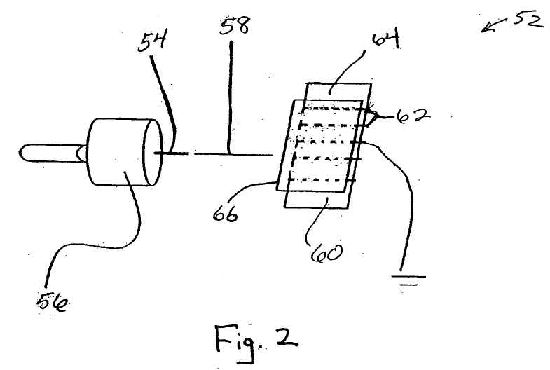

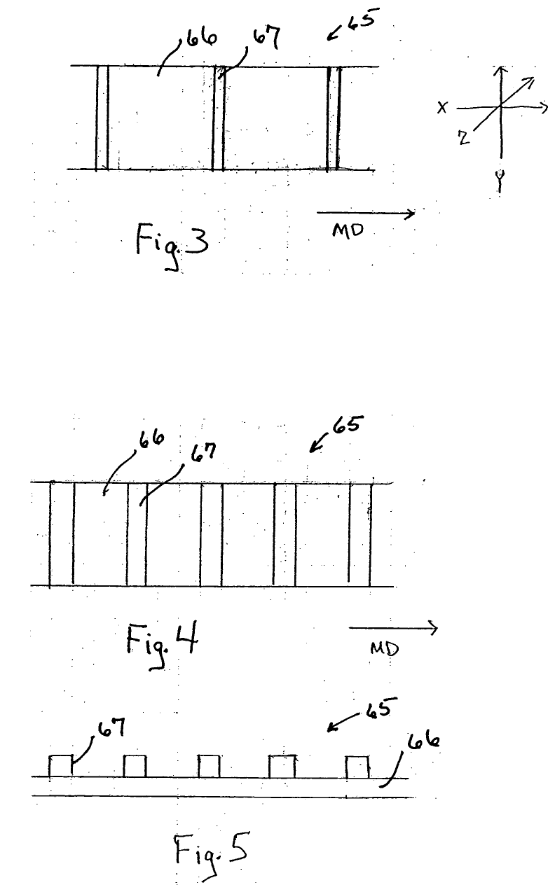

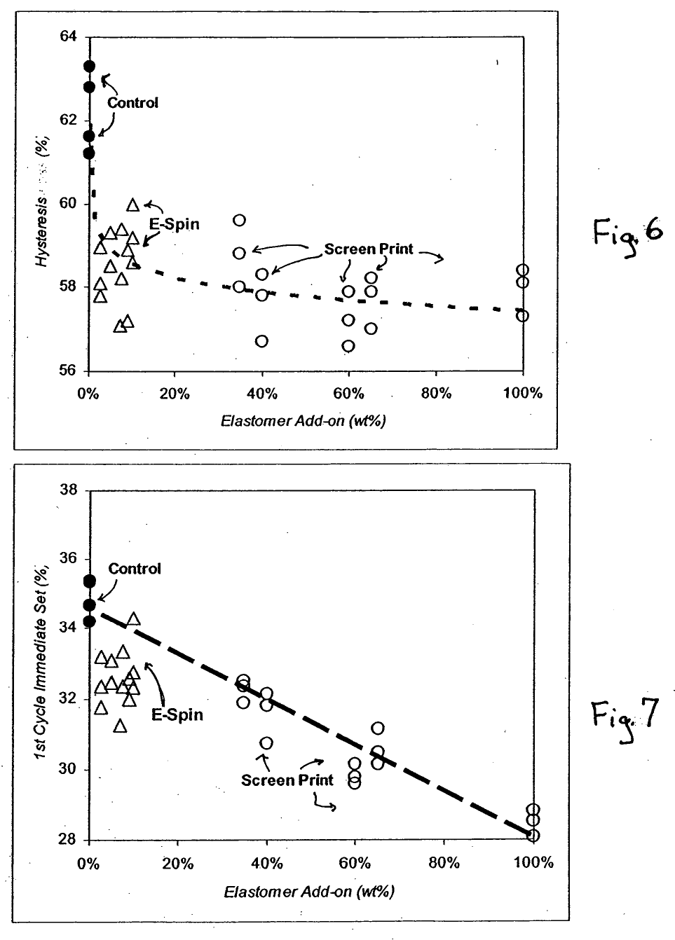

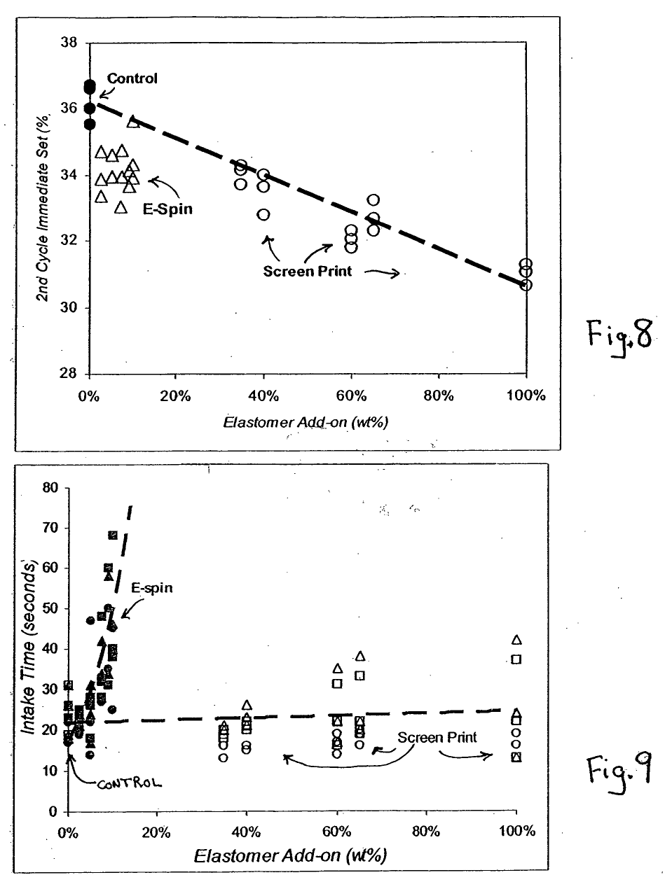

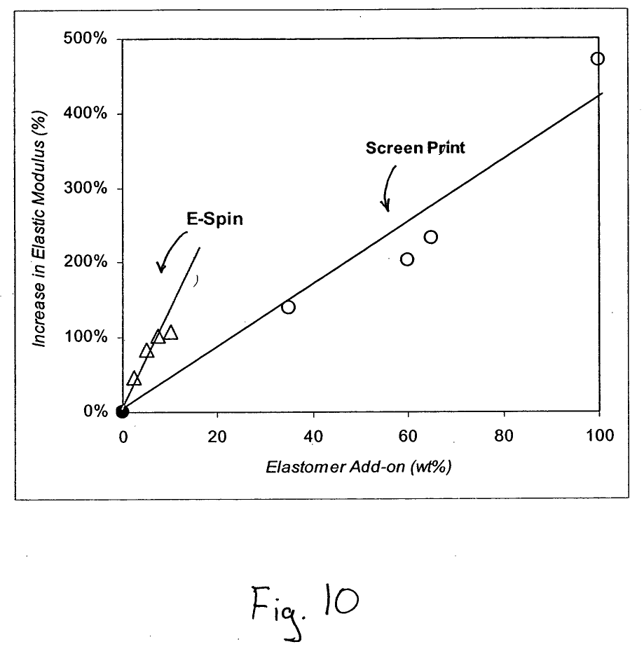

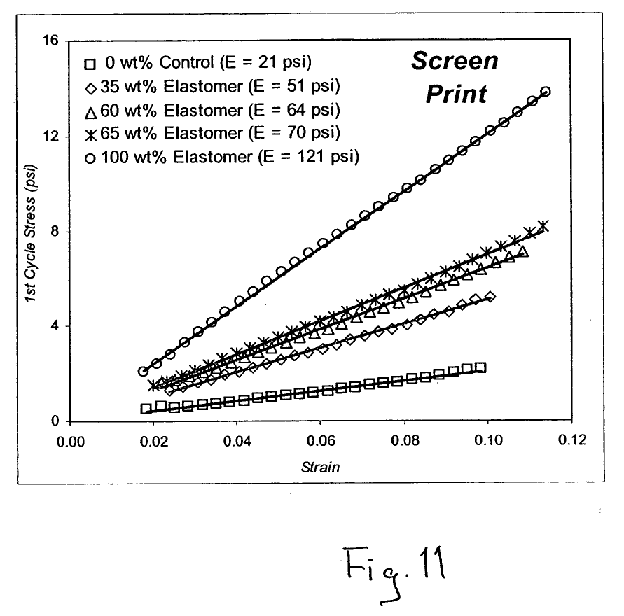

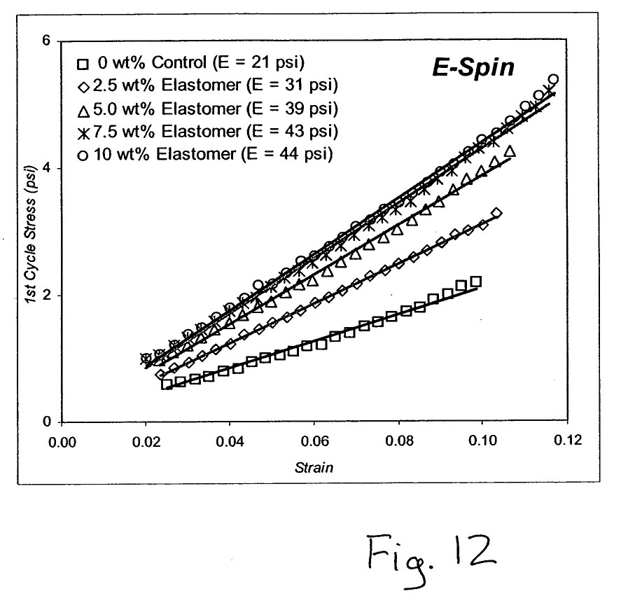

The liquid retention structure 34 provides an absorbent structure for holding and storing absorbed liquids and other waste materials, such as the shown absorbent pad composed of selected hydrophilic fibers and high-absorbency particles. The liquid retention structure 34 may also be extendible or not extendible, although it should not interfere with the expanding of the waistband or leg cuff areas. The liquid retention structure 34 is positioned and sandwiched between the liner 32 and backsheet 30 to form the diaper 20. The liquid retention structure 34 has a construction that is generally compressible, conformable, non-irritating to the wearer's skin, and capable of absorbing and retaining body exudates. A spacer layer 54 (FIG. 2) may further be provided to act as a ventilation layer to insulate the backsheet 30 from the liquid retention structure 34 to reduce the dampness of the garment at the exterior surface of the backsheet 30.

A substantially hydrophilic tissue wrapsheet is employed in the exemplary embodiment to help maintain the integrity of the fibrous structure of the liquid retention structure 34. The tissue wrapsheet is typically placed about the liquid retention structure over at least one major facing surface thereof and composed of an absorbent cellulosic material, such as creped wadding or a high wet-strength tissue that may or may not be pleated. In one aspect of the invention, the tissue wrapsheet can be configured to provide a wicking layer which helps to rapidly distribute liquid over the mass of absorbent fibers including the liquid retention structure 34. The wrapsheet material on one side of the absorbent fibrous mass may be bonded to the wrapsheet located on the opposite side of the fibrous mass to effectively entrap the liquid retention structure 34. The spacer layer and certain absorbent core coverings may be given improved retractive ability through the use of the present invention.

### PROCESSING EXAMPLES

The following examples are presented to provide a more detailed understanding of the invention. The examples are representative, and are not intended to limit the scope of the invention.

FIG. 3 and FIG. 4 show top plan views of a composite 65 with a substrate 66 and coalesced elastomer stripes 67 placed thereon. Coverage area of the coalesced elastomer stripes 67 may desirably be between 5% and 50% on the surface area of the substrate 66, also desirably between 10% and 40%, and also desirably between 13.5% and 37.5% as set forth in the exemplary materials. The X, Y and Z axes are indicated in FIG. 3. The MD is indicated by an arrow in each of FIG. 3 and FIG. 4. FIG. 5 shows a side view elevation of the Z axis of a composite 65 with a substrate 66 and coalesced elastomer stripes 67 placed thereon. It will be appreciated by the person of skill in the art that the boundaries of the stripes need not necessarily be regular as shown in the illustrated examples to remain within the spirit of the present invention.

Embodiments of the present invention with a screen print application of coalesced elastomer to the control material as a substrate comprised the control material and an elastomer of a styrenic block copolymer (SBC) tradenamed Kraton 1101D, a styrene butadiene styrene copolymer (SBS), from Kraton Polymers of Houston Tex., in a 50% solids water-based emulsion. The control material is an extendible 0.6 osy, 35% necked, nonwoven polypropylene spunbond without the addition of elastomers. Lines of the emulsion were applied in 1 mm wide lines of coalesced elastomer printed at 1 cm spacings along the CD direction of the control material using a cardboard template. The CD was the direction of extensibility of the substrate and the longitudinal direction of the stripes of coalesced elastomer 67 were placed substantially in the CD axis or direction of extensibility. “In the direction of extensibility,” “along the direction of extensibility” or the like is intended herein to include placement parallel to that axis or up to 45° therefrom. Various methods of screen printing or its equivalents are assumed to be well known in the art and will not be further elaborated on herein. Coalesced elastomerics were printed until add on weight percents of 35 percent, 40 percent, 60 percent, 65 percent and 100 percent were achieved. “Weight percent” as referred to herein can be derived by dividing the amount of add-on by the original substrate weight. For example, a 10 gram (g) substrate with 1 g of add-on will have an add-on weight percent of 10%.

Embodiments of the present invention with an electrospinning application of microfibers to the control material comprised the control material and an elastomer of Kraton 1101D SBS copolymer in a 14 wt % solids solution with a 75%/25% tetrahydrofuran (THF)/dimethylformamide (DMF) organic solvent.

Referring to FIG. 2, the electrospinning apparatus 52 metered the elastomer solution through a metal orifice (OD=⅛ inch) 54 at a rate of 0.1 ml/min. A charge was applied to the orifice 54 by a 15 kV power supply 56. The charged elastomer solution 58 was then drawn to a small diameter (500 nm-3 micron), i.e. microfiber size by placing a grounded plate 60 about 10 inches from the orifice 54. The nonwoven substrate 66 was then placed over the grounding plate 60 to receive the charged elastomer solution or microfiber 58. The grounding plate 60 had conductive areas 62 and nonconductive areas 64 in the desired pattern for fiber deposition. The pattern used was a pinstripe pattern consisting of 4 mm wide lines of coalesced elastomer deposition spaced about 1 cm apart in the CD and oriented longitudinally on the extensible axis (CD) of the control nonwoven. It will be appreciated that other patterns may be used if deemed necessary or desirable within the spirit of the present invention. The control nonwoven was placed over the grounding plate and the microfibers were deposited to attain coalesced elastomer add-on levels of 2.5, 5.0, 7.5, 9.0, and 10.0 wt %.

Variants of elastomeric materials suitable for use with the present invention may occur to the person having ordinary skill in the art upon gaining an understanding of the invention as presented herein and may desirably include without limitation polyurethane elastomers, polyolefin elastomers, thermoplastic ether elastomers and thermoplastic ester elastomers. Likewise, the solvents and elastomeric solutions given are not necessarily to be taken as ideal or definitive of the invention and may be adapted. Other patterns may be found desirable to provide for optimized or customized retractive performance. Also, in some embodiments the charged elastomer solution may be presented as droplets rather than as microfibers if such an application is found to be efficacious.

Testing Data is presented in Tables 1-4, as well as graphically in FIGS. 6-12. In Tables 1 and 2, cycle testing (as set forth below) loads in gram force at 30% and 50% of the Up (extension) and Down (retraction) cycles for first and second cycles are reported under the column headings Load @ 30 Up Cyc 1, Load @ 50 Up Cyc 1, Load @ 30 Dn Cyc 1, Load @ 50 Dn Cyc 1; and Load @ 30 Up Cyc 2, Load @ 50 Up Cyc 2, Load @ 30 Dn Cyc 2, and Load @ 50 Dn Cyc 2, respectively. Also given are columns for TEA (total energy absorption) figures in kilograms/millimeter (kg/mm) as derived from the areas under the load versus extension curves for the samples for extension (Ext), and retraction (Ret) at Cycle 1 (Cyc 1) and Cycle 2 (Cyc 2). Also given are columns for the hysteresis percentages (% Hyster Loss) at Cycle 1 (Cyc 1) and Cycle 2 (Cyc 2) and Immediate Set percentage (Immed Set %) at Cycle 1 (Cyc 1) and Cycle 2 (Cyc 2) and Load Loss at 50% extension in gram force. Hysteresis may be determined from the below-described cycle test whereby a sample is stretched to a desired elongation and recovered to zero extension. Loading energy is calculated by the area under the loading curve and unloading energy is calculated by the area under the unloading curve. Percentage hysteresis is then calculated as (the loading energy minus the unloading energy) divided by the loading energy and multiplied by 100. Point hysteresis may similarly be determined from (extension tension at a given elongation minus retraction tension at the same elongation) divided by extension tension at the same elongation and multiplied by 100. In Tables 3 and 4 it can be seen that the percentage of hysteresis, immediate set for both first and second cycle testing, and modulus of elasticity improvements all show improvement in the retractive ability of the substrate over the control material. The testing procedures are further described below.

Cycle Testing

The materials were tested using a cyclical testing procedure to determine hysteresis and percent set. In particular, a 3 cycle testing was utilized to a 50 percent defined elongation. For this test, the sample size was 3 inches in the MD by 6 inches in the CD. The grip size was 3 inches in width. The initial grip separation was 4 inches. The samples were loaded such that the cross-direction of the sample was in the vertical, or cycling, direction. A preload tension of approximately 10-15 grams was set. The test pulled the sample at 20 inches/min (500 mm/min) to a 50 percent elongation, i.e., 2 inches in addition to the 4 inch gap, and then immediately without pause returned to the zero point, i.e., the 4 inch gauge separation. The test repeated the cycle for a sample up to 3 times. The sample was then extended to failure, i.e. its breaking point. In-process testing (resulting in the data in this application) was done as a 3-cycle test. The results of the test data are all from the first and to second cycles. The testing was done on a Sintech Corp. 2/S constant rate extension testing frame with an MTS RENEW controller using TESTWORKS 4.07b software from MTS Systems Corporation of Eden Prairie, Minn. The tests were conducted under ambient temperature and humidity room conditions. Intermediate set was determined from the length of the sample on the return or down cycle when the sample had reached zero tension.

Fluid Intake and Flowback Evaluation (FIFE) Testing:

Generally, this test has been designed to measure the absorbency/penetration time of an absorbent article, such as an infant diaper. The absorbency/penetration time (in seconds) is measured using a stopwatch and visually determining the length of time required to absorb simulated urine voidings.

Equipment & Materials Used in the FIFE Test Include

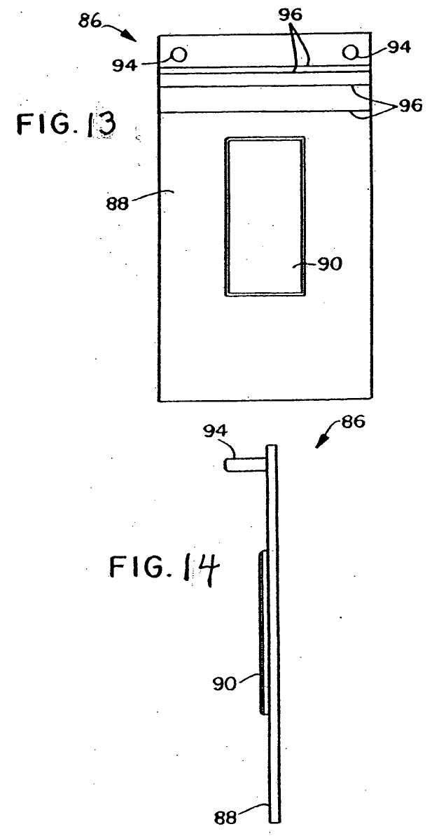

1. FIFE Boards. As representatively shown in FIGS. 13 and 14, bottom FIFE board 86 includes a rectangular shaped base member 88 and a smaller, rectangular shaped platform member 90. The base member has an overall length (top to bottom of the figure) of 35.6 cm (14 in), an overall side-to-side width of 20.3 cm (8 in) and a thickness of 0.86 cm (0.34 in). Platform member 90 has a length of 15.2 cm (6 in), a width of 10.2 cm (4 in) and a thickness of 0.56 cm (0.22 in). The platform member is centered onto the top surface of base 88 and secured in place, such as by adhesive bonding. The four, peripheral top edges of platform 90 are shaped with a 0.15 cm (0.06 inch) by 45° chamfer. Rectangular base 88 includes a pair of 1.27 cm (0.5 inch) diameter, cylindrical rods 94 which are press fitted into mating holes and secured in place with suitable attachment means, such as adhesive bonding. The center of each rod is positioned 1.9 cm (0.75 in) from the top, end edge of the base member, and 1.9 cm (0.75 in) from the immediately adjacent side edge of the base member. The rods extend about 4.14 cm (about 1.63 in) above the surface of the base member, and the uppermost exposed edges of rods 94 are rounded with a contour radius of about 0.41 cm (about 0.16 inches). A series of four reference lines 96 are scribed into the top surface of base 88 and extend laterally across the width of the base member. The scribe lines are parallel and spaced from the top, end edge of base 88 by distances of 3.18 cm (1.25 in), 3.81 cm (1.50 in), 5.08 cm (2.00 in) and 7.62 cm (3 in), respectively. The components of bottom FIFE board 86 are composed of a suitable water resistant material, such as Lexan plastic.

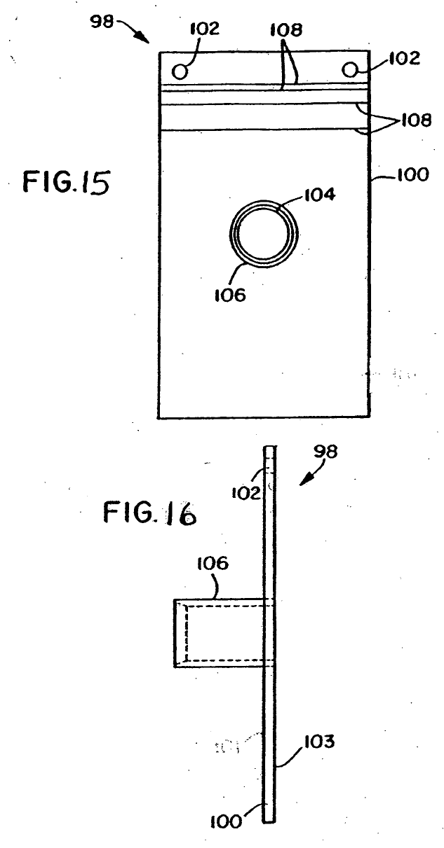

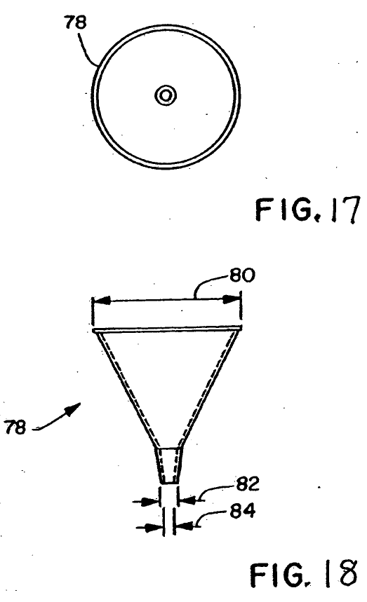

Referencing FIGS. 15-16, top FIFE board 98 includes a top plate 100 and a cylindrical tube 106 which extends generally perpendicular from the plane defined by uppermost, top surface of the top plate. Top plate 100 is generally rectangular in shape and is sized with substantially the same length, width and thickness as bottom FIFE board 86. The top plate includes a pair of 1.35 cm (0.53 inch) diameter through holes 102 which are located adjacent the top edge of plate 100 and configured to slip over rods 94 in bottom FIFE board 86 to appropriately locate top FIFE board 98 in a substantially congruent, coextensive position over bottom FIFE board 86. A series of four reference lines 108 are inscribed into a top surface of plate 100 and extend linearly in the transverse direction across the width of the top plate. The scribe lines are parallel and spaced from the top, end edge of base 88 by distances of 3.18 cm (1.25 in), 3.81 cm (1.50 in), 5.08 cm (2.00 in) and 7.62 cm (3 in), respectively. The medial section of top plate 100 includes a circular hole which is sized to accept the placement of cylindrical tube 106. Tube 106 has a 6.35 cm (2.5 inch) outside diameter, a 5.08 cm (2.0 inch) inside diameter and an overall length of 9.53 cm (3.75 in). The tube press fitted and attached in place within center hole 104 by suitable attachment means, such as adhesive bonding. Hole 104 is centered with respect to both the length and width of the top plate. Tube 106 projects generally perpendicular the top of plate 100 and extends through the of the plate to protrude a small distance of about 0.76 mm (about 0.03 inches) past bottom surface 103 of plate 100. The upper, entrance edge of tube 106 has an internal chamfer which generally matches the conical shape of the associated funnel representatively shown in FIGS. 17-18. Similar to the components of bottom FIFE board 86, the components of top plate 100 are composed a suitable water resistant material, such as Lexan plastic.

2. A 113.4 gram (four ounce) Funnel; see FIGS. 16A and 16B. Funnel 78 has inlet diameter 80 of 8.26 cm (3.25 in), a funnel throat diameter 82 of 1.113 cm (0.438 in) and a spout outlet diameter 84 of 0.635 cm (0.25 in). The given measurements are inside diameters.

Testing Procedure:

1. Place the specimen on the bottom FIFE board.

2. Align the specimen so the target zone is in the center of the 5.1×15.2 cm (3×6 inch) raised platform. The target zone is along the lengthwise center of the test article and is located a specific number of centimeters (inches) from the front edge of the specimen. The number of inches from the front edge of the specimen is determined by the size of the article, as follows:

3. Place the top FIFE board over the target, making sure there are no apparent wrinkles in the liner under the Board. Press lightly on the board to impress the cylinder ridge (on underside of board) into the specimen.

4. Place the funnel into the cylinder. The funnel must be perpendicular to the specimen and in the center of the target zone area. This is determined by sighting through the end of the funnel.

5. Measure the appropriate amount of testing liquid using the dispenser and dispense into the beaker.

6. Pour the liquid fast as possible without overflowing the funnel from the beaker into the funnel and onto the target area. Start the stopwatch when the liquid hits the funnel.

7. As soon the funnel is empty, remove it.

8. Observe the liquid intake through the cylinder top. Record the time to the nearest second the moment no liquid is visible on the specimen surface. Allow the stop watch to continue running.

9. Using the timer, wait exactly one minute from the starting time of step 6. Then repeat steps 5 through 8 for the second liquid intake.

10. Using the timer, wait exactly two minutes from the starting time of step 6. Then repeat steps 5 through 8 for the third liquid intake.

As seen in the graph of FIG. 6, a graph of coalesced elastomer add-on weight percent on the X axis against hysteresis percent on the Y axis, the control material with 0.0% coalesced elastomer add-on, indicated as dots, showed a hysteresis of between about 61.5% to about 63.5%. The data points for weight percents below 20%, i.e., for electrospinning embodiments, indicated as triangles, show a hysteresis ranging from between about 57% to about 60%. These electrospinning data points are slightly above the 35%, 40%, 60%, 65%, and 100% add-ons for screen printing, indicated as circles, which show a slight downward trend from the electrospinning median of about 59% to a median of about 58%. Thus an improvement in hysteresis is demonstrated over the control samples.

As seen in the graph of FIG. 7, a graph of coalesced elastomer add-on weight percent on the X axis against a cycle test first cycle test immediate set on the Y axis, the control material with 0.0% coalesced elastomer add-on, indicated as dots, showed a first cycle immediate set of between about 34.0 % to about 35.5%. The data points for weight percents below 20% for electrospinning embodiments, indicated as triangles, show a first cycle test immediate set ranging from between about 31.5% to about 34.5% with a median of about 33%. The data points for the add-ons for screen printing, indicated as circles, show between about 31%-33% set for 35%-40% add-ons, about 30%-32% set for the 60%-65% add-ons, to about 28%-29% set for the 100% add-ons. A consistent downward slope, or improvement, is indicated for first cycle immediate set over the control material as the add on weight increases.

As seen in the graph of FIG. 8, a graph of coalesced elastomer add-on weight percent on the X axis against a cycle test second cycle test immediate set on the Y axis, the control material with 0.0% coalesced elastomer add-on, indicated as dots, showed a second cycle immediate set of between about 35.5% to about 37%. The data points for weight percents below 20% for electrospinning embodiments, indicated as triangles, generally show a second cycle test immediate set ranging from between about 33% to about 35% with a median of about 34%. The data points for the add-ons for screen printing, indicated as circles, show between about 33%-35% set for 35%-40% add-ons, about 32%-34% set for the 60%-65%, to about 31% set for the 100% add-ons. A consistent downward slope, or improvement, is indicated for second cycle immediate set over the control material as the add on weight increases.

In the graph of FIG. 9, a graph of coalesced elastomer add-on weight percent on the X axis against a three insult FIFE intake test time in seconds on the Y axis, is shown. First intake data points are indicated as circles, second intake data points are indicated as squares and third intake data points are indicated as triangles. While intake times for some samples of weight percent add-ons below 20%, i.e., electrospinning embodiments, generally range around the 15 second—30 second time span indicated for the data points for the 0% control add-on and the screen printing embodiments at 35%-40%, 60%-65%, and 100% add-ons, in some samples a large increase in intake time for sequential insults was observed. Without being bound by theory, it is believed that in some samples of electrospinning embodiments, stripe formation was not as clearly defined due to the insulating effect of previous fiber deposition working against clearly defined electrospinning deposition of fibers near the end of the deposition process. It is believed this may be cured by increasing the difference in potential between the charged fibers and the plate underlying the nonwoven substrate web to minimize the drifting of fibers during deposition. However, it is believed to be adequately shown that in most embodiments of the present invention, as evidenced by the test samples, no significant deleterious effect on fluid intake time was had. Thus, composite materials according to the present invention are believed to provide adequate fluid intake properties if used as liner material for a disposable absorbent garment.

From the graphs of FIGS. 10 -12 and Tables 3 and 4 it can be seen that there is a substantially linear increase in modulus of elasticity for the composite material in relation to the amount of elastomer add-on for the embodiments of the present invention over that of the unimproved control material. As seen in FIGS. 10 and 12, for electrospinning embodiments with add-on weight percents of between 2.5% to 10% there is an increase in the modulus of between 40% and 100%. As seen in FIGS. 10 and 11, for screen printing embodiments with add-on weight percents of between 35% to 100% there is an increase in the modulus of between 140% and 470%.

Thus it can be seen that the present invention has presented a useful and economical means of providing substrates, and especially nonwoven substrates suitable for use in disposable absorbent articles or garments, with improved retraction while retaining fluid permeability.

While the invention has been described in detail with respect to specific embodiments thereof, it will be appreciated that those skilled in the art, upon attaining an understanding of the foregoing may readily conceive of alterations to, variations of, and equivalents to these embodiments. Accordingly, the scope of the present invention should be assessed as that of the appended claims and any equivalents thereto.

TABLE 1

| | | | Load @ 30 | Load @ 50 | Load @ 30 | Load @ 50 | TEA (Ext) | TEA (Pet) | % Hyster | Load @ 30 |
|---|---|---|---|---|---|---|---|---|---|---|
| | Spam | [symbol] add-on | Up Cyc1 | Up Cyc1 | Dn Cyc1 | Dn Cyc1 | Cyc1 | Cyc1 | Loss | Up Cyc2 |
| | No. | wt % | gf | gf | gf | gf | kg [symbol] mm | kg [symbol] mm | Cyc1% | gf |
| Control | 1 | 0.0% | 335 | 831 | −18 | 678 | 0.194 | 0.033 | 82.8 | 33 |
| | 2 | 0.0% | 473 | 964 | −15 | 787 | 0.254 | 0.042 | 83.6 | 58 |
| | 3 | 0.0% | 409 | 934 | −17 | 763 | 0.227 | 0.041 | 81.8 | 42 |
| | 4 | 0.0% | 373 | 861 | −18 | 697 | 0.208 | 0.035 | 83.2 | 34 |
| 1SP | 1 | 35.0% | 480 | 933 | −3 | 760 | 0.26 | 0.049 | 81.1 | 73 |
| | 2 | 35.0% | 539 | 1049 | −3 | 851 | 0.292 | 0.055 | 81.2 | 78 |
| | 3 | 35.0% | 601 | 1154 | 0 | 939 | 0.323 | 0.06 | 81.3 | 88 |
| 2SP | 1 | 65.0% | 516 | 1008 | 3 | 832 | 0.279 | 0.059 | 78.9 | 95 |
| | 2 | 65.0% | 618 | 1068 | 6 | 889 | 0.323 | 0.063 | 80.5 | 116 |
| | 3 | 65.0% | 670 | 1198 | 9 | 987 | 0.353 | 0.072 | 79.6 | 123 |
| 3SP | 1 | 40.0% | 629 | 1138 | 5 | 941 | 0.332 | 0.069 | 79.3 | 107 |
| | 2 | 40.0% | 550 | 1069 | −2 | 878 | 0.293 | 0.057 | 80.5 | 77 |
| | 3 | 40.0% | 520 | 953 | 0 | 790 | 0.272 | 0.052 | 80.9 | 83 |
| 4SP | 1 | 100.0% | 687 | 1142 | 19 | 930 | 0.366 | 0.069 | 81.2 | 148 |
| | 2 | 100.0% | 708 | 1192 | 18 | 983 | 0.373 | 0.072 | 80.8 | 150 |
| | 3 | 100.0% | 731 | 1184 | 23 | 976 | 0.385 | 0.073 | 80.9 | 168 |
| 5SP | 1 | 60.0% | 619 | 1133 | 9 | 926 | 0.329 | 0.065 | 80.4 | 115 |
| | 2 | 60.0% | 676 | 1154 | 11 | 950 | 0.352 | 0.068 | 80.7 | 130 |
| | 3 | 60.0% | 758 | 1339 | 13 | 1094 | 0.398 | 0.078 | 80.4 | 140 |

| | | Load @ 50 | Load @ 30 | Load @ 50 | TEA (Ext) | TEA (Pet) | % Hyster | Immed | Immed | Load Loss |
|---|---|---|---|---|---|---|---|---|---|---|
| | Spam | Up Cyc2 | Dn Cyc2 | Dn Cyc2 | Cyc2 | Cyc2 | Loss | Set % | Set % | at 50% |
| | No. | gf | gf | gf | kg [symbol] mm | kg [symbol] mm | Cyc2% | Cyc1% | Cyc2% | gf |
| Control | 1 | 767 | −22 | 655 | 0.086 | 0.032 | 63.3 | 36.387 | 36.7 | 210.974 |
| | 2 | 888 | −21 | 759 | 0.11 | 0.042 | 61.6 | 34.199 | 35.538 | 212.451 |
| | 3 | 862 | −22 | 738 | 0.103 | 0.04 | 61.2 | 34.692 | 36.025 | 210.054 |
| | 4 | 789 | −23 | 673 | 0.091 | 0.034 | 62.8 | 36.341 | 36.599 | 218.703 |
| 1SP | 1 | 848 | −10 | 726 | 0.112 | 0.045 | 59.6 | 32.512 | 34.27 | 222.332 |
| | 2 | 950 | −10 | 809 | 0.125 | 0.051 | 58.8 | 32.37 | 34.144 | 228.89 |
| | 3 | 1051 | −9 | 899 | 0.14 | 0.059 | 58 | 31.867 | 33.689 | 220.98 |
| 2SP | 1 | 916 | −7 | 787 | 0.128 | 0.054 | 58.2 | 31.151 | 33.206 | 219.291 |
| | 2 | 980 | −5 | 842 | 0.142 | 0.06 | 57.9 | 30.488 | 32.668 | 211.628 |
| | 3 | 1092 | −4 | 941 | 0.158 | 0.068 | 57 | 30.132 | 32.298 | 214.511 |
| 3SP | 1 | 1041 | −6 | 899 | 0.147 | 0.064 | 56.7 | 30.714 | 32.775 | 210.6 |
| | 2 | 977 | −10 | 833 | 0.13 | 0.055 | 57.8 | 32.119 | 33.98 | 220.908 |
| | 3 | 873 | −9 | 750 | 0.119 | 0.06 | 58.3 | 31.793 | 33.639 | 212.73 |
| 4SP | 1 | 1016 | 4 | 875 | 0.155 | 0.064 | 58.4 | 28.52 | 31.069 | 233.821 |
| | 2 | 1071 | 2 | 922 | 0.164 | 0.07 | 57.3 | 28.817 | 31.26 | 226.97 |
| | 3 | 1062 | 6 | 906 | 0.168 | 0.07 | 58.1 | 28.065 | 30.633 | 234.636 |
| 5SP | 1 | 1031 | −2 | 890 | 0.146 | 0.063 | 57.2 | 30.133 | 32.303 | 215.08 |
| | 2 | 1052 | −2 | 897 | 0.154 | 0.065 | 57.9 | 29.761 | 32.053 | 222.192 |
| | 3 | 1219 | −1 | 1052 | 0.177 | 0.077 | 56.6 | 29.602 | 31.806 | 213.999 |

TABLE 2

| | | | Load @ 30 | Load @ 50 | Load @ 30 | Load @ 50 | TEA (Ext) | TEA (Pet) | % Hyster | Load @ 30 |
|---|---|---|---|---|---|---|---|---|---|---|
| | Spam | [symbol] add-on | Up Cyc1 | Up Cyc1 | Dn Cyc1 | Dn Cyc1 | Cyc1 | Cyc1 | Loss | Up Cyc2 |
| | No. | wt % | gf | gf | gf | gf | kg [symbol] mm | kg [symbol] mm | Cyc1% | gf |
| 6 ES | 1 | 10.0% | 262 | 864 | −7 | 694 | 0.183 | 0.039 | 78.5 | 40 |
| | 2 | 10.0% | 581 | 1077 | −8 | 883 | 0.301 | 0.052 | 82.6 | 86 |
| | 3 | 10.0% | 614 | 1161 | −6 | 956 | 0.319 | 0.06 | 81.3 | 90 |
| 7 ES | 1 | 5.0% | 162 | 664 | −10 | 536 | 0.129 | 0.027 | 79.2 | 25 |
| | 2 | 5.0% | 396 | 861 | −6 | 707 | 0.22 | 0.044 | 80.1 | 59 |
| | 3 | 5.0% | 498 | 934 | −3 | 767 | 0.262 | 0.05 | 80.8 | 76 |
| 8 ES | 1 | 2.5% | 611 | 1107 | −4 | 920 | 0.317 | 0.059 | 81.4 | 88 |
| | 2 | 2.5% | 549 | 1057 | 0 | 867 | 0.29 | 0.056 | 80.6 | 86 |
| | 3 | 2.5% | 447 | 960 | −6 | 787 | 0.248 | 0.05 | 79.7 | 61 |
| 9 ES | 1 | 7.5% | 531 | 1037 | −3 | 846 | 0.285 | 0.055 | 80.7 | 76 |
| | 2 | 7.5% | 601 | 1113 | 2 | 919 | 0.311 | 0.062 | 80 | 101 |
| | 3 | 7.5% | 444 | 903 | −7 | 742 | 0.239 | 0.043 | 82 | 61 |
| 10 ES | 1 | 9.0% | 512 | 954 | −1 | 777 | 0.27 | 0.051 | 80.9 | 83 |
| | 2 | 9.0% | 493 | 1084 | −4 | 890 | 0.278 | 0.059 | 78.9 | 70 |
| | 3 | 9.0% | — | — | — | — | — | — | — | — |

| | | Load @ 50 | Load @ 30 | Load @ 50 | TEA (Ext) | TEA (Pet) | % Hyster | Immed | Immed | |
|---|---|---|---|---|---|---|---|---|---|---|
| | Spam | Up Cyc2 | Dn Cyc2 | Dn Cyc2 | Cyc2 | Cyc2 | Loss | Set % | Set % | Load Loss |
| | No. | gf | gf | gf | kg [symbol] mm | kg [symbol] mm | Cyc2% | Cyc1% | Cyc2% | at 50% gf |
| 6 ES | 1 | 782 | −11 | 667 | 0.09 | 0.037 | 59.2 | 34.324 | 35.629 | 218.854 |
| | 2 | 992 | −16 | 844 | 0.132 | 0.053 | 60 | 32.762 | 34.328 | 216.312 |
| | 3 | 1068 | −13 | 919 | 0.143 | 0.059 | 58.6 | 32.315 | 33.914 | 207.838 |
| 7 ES | 1 | 611 | −14 | 514 | 0.066 | 0.025 | 62 | 35.958 | 37.13 | 226.497 |
| | 2 | 797 | −10 | 680 | 0.102 | 0.042 | 59.3 | 33.075 | 34.616 | 211.206 |
| | 3 | 859 | −9 | 738 | 0.116 | 0.048 | 58.5 | 32.447 | 33.969 | 210.292 |
| 8 ES | 1 | 1025 | −12 | 879 | 0.138 | 0.058 | 57.8 | 32.348 | 33.896 | 205.988 |
| | 2 | 971 | −6 | 827 | 0.132 | 0.055 | 58.1 | 31.77 | 33.37 | 217.485 |
| | 3 | 886 | −12 | 761 | 0.112 | 0.046 | 58.9 | 33.194 | 34.722 | 207.293 |
| 9 ES | 1 | 956 | −8 | 814 | 0.126 | 0.053 | 58.2 | 32.363 | 33.964 | 214.699 |
| | 2 | 1027 | −6 | 885 | 0.144 | 0.062 | 57.1 | 31.253 | 33.019 | 204.361 |
| | 3 | 836 | −12 | 712 | 0.107 | 0.043 | 59.4 | 33.349 | 34.768 | 211.468 |
| 10 ES | 1 | 873 | −8 | 744 | 0.118 | 0.049 | 58.9 | 31.996 | 33.648 | 219.88 |
| | 2 | 1004 | −9 | 852 | 0.131 | 0.066 | 57.2 | 32.586 | 34.158 | 214.45 |
| | 3 | — | — | — | — | — | — | — | — | — |

TABLE 3
Mechanical Properties of Screen Printed Materials

| | Elastomer | % Hyster | % Reduction | Immed | % Reduction | Immed | % Reduction | Modulus of | % Improve |
|---|---|---|---|---|---|---|---|---|---|
| | add-on | Loss | vs | Set % | vs | Set % | vs | Elasticity | vs |
| | wt % | Cyc 1% | Control % | Cyc 1% | Control % | Cyc2% | Control % | psi | Control % |
| Control | 0% | 83% | N/A | 35% | N/A | 36% | N/A | 21 | N/A |
| Sample1sp | 35% | 81% | 2% | 32% | 10% | 34% | 6% | 51 | 140% |
| Sample3sp | 40% | 80% | 3% | 32% | 11% | 33% | 7% | — | — |
| Sample5sp | 60% | 81% | 3% | 30% | 16% | 32% | 11% | 64 | 200% |
| Sample2sp | 65% | 80% | 4% | 31% | 14% | 33% | 9% | 70 | 230% |
| Sample4sp | 100% | 81% | 2% | 28% | 20% | 31% | 14% | 121 | 470% |

TABLE 4 [symbol]
hanical Properties of E-Spin Materials

| | Elastomer | % Hyster | % Reduction | Immed | % Reduction | Immed | % Reduction | Modulus of | % Improve |
|---|---|---|---|---|---|---|---|---|---|
| | add-on | Loss | vs | Set % | vs | Set % | vs | Elasticity | vs |
| | wt % | Cyc 1% | Control % | Cyc 1% | Control % | Cyc2% | Control % | psi | Control % |
| Control | 0% | 83% | N/A | 35% | N/A | 36% | N/A | 21 | N/A |
| Sample8es | 2.5% | 81% | 3% | 32% | 8% | 34% | 6% | 31 | 40% |
| Sample7es | 5% | 80% | 3% | 34% | 4% | 35% | 2% | 39 | 80% |
| Sample9es | 7.5% | 81% | 2% | 32% | 9% | 34% | 6% | 43 | 100% |
| Sample10es | 9% | 80% | 4% | 32% | 10% | 34% | 6% | — | — |
| Sample6es | 10% | 81% | 2% | 33% | 6% | 35% | 4% | 44 | 100% |

1. A retractive composite web comprising:
a) an extensible, fluid permeable substrate having at least one direction of extensibility in an XY plane;
b) a pattern of untensioned coalesced elastomeric stripes contained on an XY plane surface of the substrate in an add-on amount of between about 2.5 weight percent to about 100 weight percent of the composite web;
c) each longitudinal axis of the coalesced elastomeric stripes being oriented in the direction of substrate extensibility; and
d) the retractive composite being fluid permeable.

2. The retractive composite web of claim 1 wherein the pattern is a regular pattern of 1 mm wide stripes spaced 1 cm apart.

3. The retractive composite web of claim 1 wherein the pattern is a regular pattern of 4 mm wide stripes spaced 1 cm apart.

4. The retractive composite web of claim 1 wherein the coverage area of stripes is between about 10.0 and about 40.0 percent of the surface area of the substrate.

5. The retractive composite web of claim 1 wherein the coalesced elastomer is in an add on amount of between about 2.5 weight percent to about 10.0 weight percent.

6. The retractive composite of claim 5 having a modulus of elasticity of between about 31 psi and about 44 psi.

7. The retractive composite web of claim 1 wherein the coalesced elastomer is in an add on amount of between about 35.0 weight percent to about 40.0 weight percent.

8. The retractive composite of claim 7 having a modulus of elasticity of about 51 psi.

9. The retractive composite web of claim 1 wherein the coalesced elastomer is in an add on amount of between about 60.0 weight percent to about 65.0 weight percent.

10. The retractive composite of claim 9 having a modulus of elasticity of between about 64 psi and about 70 psi.

11. The retractive composite web of claim 1 wherein the coalesced elastomer is in an add on amount of about 100.0 weight percent.

12. The retractive composite of claim 11 having a modulus of elasticity of about 121 psi.

13. The retractive composite web of claim 1 wherein the coalesced elastomer is in an add on amount of up to about 100.0 weight percent.

14. The retractive composite web of claim 1 wherein the extensible, fluid permeable substrate has a basis weight of about 0.6 osy.

15. The retractive composite web of claim 14 wherein the coalesced elastomer is in an add on amount of between about 2.5 weight percent to about 10.0 weight percent.

16. The retractive composite of claim 15 having a modulus of elasticity of between about 31 psi and about 44 psi.

17. The retractive composite web of claim 14 wherein the coalesced elastomer is in an add on amount of between about 35.0 weight percent to about 40.0 weight percent.

18. The retractive composite of claim 17 having a modulus of elasticity of about 51 psi.

19. The retractive composite web of claim 14 wherein the coalesced elastomer is in an add on amount of between about 60.0 weight percent to about 65.0 weight percent.

20. The retractive composite of claim 19 having a modulus of elasticity of between about 64 psi and about 70 psi.

21. The retractive composite web of claim 14 wherein the coalesced elastomer is in an add on amount of about 100.0 weight percent.

22. The retractive composite of claim 21 having a modulus of elasticity of about 121 psi.

23. The retractive composite web of claim 1 wherein the extensible, fluid permeable substrate is a polypropylene spunbond nonwoven.

24. The retractive composite web of claim 1 wherein the extensible, fluid permeable substrate is necked from about 35% to about 50%.

25. The retractive composite web of claim 1 wherein the coalesced elastomeric stripes are provided in an amount sufficient to provide less than about 81.8% first cycle hysteresis.

26. The retractive composite web of claim 1 wherein the coalesced elastomeric stripes are provided in an amount sufficient to provide less than about 61.2% second cycle hysteresis.

27. The retractive composite web of claim 1 wherein the coalesced elastomeric stripes are provided in an amount sufficient to provide less than about 35.0% first cycle immediate set.

28. The retractive composite web of claim 1 wherein the coalesced elastomeric stripes are provided in an amount sufficient to provide less than about 36.0% second cycle immediate set.

29. The retractive composite web of claim 1 wherein the coalesced elastomeric stripes are provided in an amount sufficient to provide a modulus of elasticity greater than 21 psi.

30. The retractive composite web of claim 1 wherein the composite has a fluid intake time of between about 15 seconds to about 30 seconds for each cycle of a 3 cycle FIFE test.

31. The retractive composite web of claim 1 wherein the elastomer is a styrenic block copolymer.

32. The retractive composite web according to of claim 1 wherein the extensible, fluid permeable substrate is a necked nonwoven web.

33. A retractive composite web comprising:
a) an extensible, fluid permeable substrate having at least one direction of extensibility in an XY plane, the extensible, fluid permeable substrate being a spunbond nonwoven having a basis weight of about 0.6 osy;
b) a pattern of untensioned coalesced elastomeric stripes contained on an XY plane surface of the substrate in an add-on amount of between about 2.5 weight percent to about 100 weight percent of the composite web;
c) each longitudinal axis of the coalesced elastomeric stripes being oriented in the direction of substrate extensibility;
d) the retractive composite being fluid permeable, having a hysteresis of less than 83%, having less than about 35.0% first cycle immediate set, having less than about 36.0% second cycle immediate set, having a modulus of elasticity greater than 21 psi, and having a fluid intake time of less than 30 seconds for each cycle of a 3 cycle FIFE test.

34. The retractive composite web of claim 33 further having a modulus of elasticity of between about 31 psi and about 44 psi.

35. The retractive composite web of claim 33 further having a modulus of elasticity of between about 51 and about 121 psi.

36. A method of producing a retractive composite web comprising:
a) providing an extensible, fluid permeable substrate having a basis weight and at least one direction of extensibility in an XY plane;
b) applying a pattern of untensioned coalesced elastomeric stripes on an XY plane surface of the substrate in an add-on amount of between about 2.5 weight percent to about 100 weight percent of the composite web with each longitudinal axis of the coalesced elastomeric stripes oriented substantially along the direction of substrate extensibility; and
c) whereby the extensible, non-retractive, fluid permeable substrate is made retractive composite while remaining fluid permeable.

37. The method of producing a retractive composite web of claim 36 further comprising:
necking the substrate to produce at least one direction of extensibility in an XY plane of the substrate.

38. The method of producing a retractive composite web of claim 36 further comprising:
applying the pattern of untensioned coalesced elastomeric stripes by printing an aqueous emulsion of coalesced elastomers onto the substrate.

39. The method of producing a retractive composite web of claim 36 further comprising:
applying the pattern of untensioned coalesced elastomeric stripes by electrospinning at least one coalesced elastomeric fiber onto the substrate.

40. The method of producing a retractive composite web of claim 36 further comprising:
applying the pattern of untensioned coalesced elastomeric stripes by electrospinning at least one coalesced elastomeric fiber onto the substrate.

41. The method of producing a retractive composite web of claim 36 further comprising:
applying the pattern of untensioned coalesced elastomeric stripes by electrospinning elastomeric droplets onto the substrate.

42. The method of producing a retractive composite web of claim 36 further comprising:
applying the coalesced elastomer as a 50% solids emulsion.

43. The method of producing a retractive composite web of claim 36 further comprising:
applying the coalesced elastomer as a solution of Kraton 1101D SBS copolymer in a 14 wt % solids solution with a 75%/25% tetrahydroftiran (THF)/dimethylformamide (DMF) organic solvent.

44. The method of producing a retractive composite web of claim 36 further comprising:
applying the coalesced elastomer as a regular pattern of 1 mm wide stripes spaced 1 cm apart.

45. The method of producing a retractive composite web of claim further comprising:
applying the coalesced elastomer as a regular pattern of 4 mm wide stripes spaced 1 cm apart.

46. The method of producing a retractive composite web of claim 36 further comprising:
applying the coalesced elastomer in a coverage area of stripes between about 5.0 and about 50.0 percent of the surface area of the substrate.

47. The method of producing a retractive composite web of claim 36 further comprising:
applying the coalesced elastomer in an add on amount of between about 2.5 weight percent to about 10.0 weight percent.

48. The method of producing a retractive composite web of claim 36 further comprising:
applying the coalesced elastomer in an add on amount of between about 35.0 weight percent to about 40.0 weight percent.

49. The method of producing a retractive composite web of claim 36 further comprising:
applying the coalesced elastomer in an add on amount of between about 60.0 weight percent to about 65.0 weight percent.

50. The method of producing a retractive composite web of claim 36 further comprising:
applying the coalesced elastomer in an add on amount of about 100.0 weight percent.

51. The method of producing a retractive composite web of claim 36 further comprising:
applying the coalesced elastomer in an add on amount of up to about 100.0 weight percent.

52. The method of producing a retractive composite web of claim 36 further comprising:
supplying the extensible, fluid permeable substrate in a basis weight of about 0.6 osy.

53. The method of producing a retractive composite web of claim 36 further comprising:
supplying the extensible, fluid permeable substrate as a polypropylene spunbond nonwoven.

54. The method of producing a retractive composite web of claim 36 further comprising:
necking the extensible, fluid permeable substrate from about 35% to about 50% to make the substrate extensible in the cross direction.

55. A disposable absorbent article comprising:
a) a liquid permeable liner layer having a top surface and a lower surface;
b) a substantially liquid impermeable outer cover; and
c) an absorbent layer disposed between said liner layer lower surface and said outer cover;
d) at least one of the liner absorbent core and outer cover having at least one layer comprising a retractive composite having:
i) an extensible, fluid permeable substrate having at least one direction of extensibility in an XY plane;
ii) a pattern of untensioned coalesced elastomeric stripes contained on an XY plane surface of the substrate in an add-on amount of between about 2.5 weight percent to about 100 weight percent of the composite web;
iii) each longitudinal axis of the coalesced elastomeric stripes being oriented in the direction of substrate extensibility; and
iv) the retractive composite being fluid permeable.

56. The disposable absorbent garment with waist and leg cuff areas with at least one of the waist and leg cuff areas comprising a retractive composite having:
i) an extensible, fluid permeable substrate having at least one direction of extensibility in an XY plane;
ii) a pattern of untensioned coalesced elastomeric stripes contained on an XY plane surface of the substrate in an add-on amount of between about 2.5 weight percent to about 100 weight percent of the composite web;
iii) each longitudinal axis of the coalesced elastomeric stripes being oriented in the direction of substrate extensibility; and
iv) the retractive composite being fluid permeable.
